(19) United States
(12) Patent Application Publication
(10) Pub. No.: US 2025/0361565 A1
(43) Pub. Date: Nov. 27, 2025

(54) SAMPLE TRACKING AND DISCRIMINATION USING GERMLINE STRUCTURAL VARIANTS

(71) Applicant: SAGA DX, INC., Morrisville, NC (US)

(72) Inventors: Miguel Alcaide, Lund (SE); Anthony Miles George, Lund (SE); Lucia Oton, Lund (SE); Yilun Chen, Lund (SE); Christopher Rushton, Lund (SE)

(21) Appl. No.: 19/213,737

(22) Filed: May 20, 2025

Related U.S. Application Data
(60) Provisional application No. 63/650,061, filed on May 21, 2024.

(51) Int. Cl. C12Q 1/6886; C12Q 1/6809; C12Q 1/6827; C12Q 1/686; C12Q 1/6869

(52) U.S. Cl. CPC C12Q 1/6886; C12Q 1/6809; C12Q 1/6827; C12Q 1/686; C12Q 1/6869

(57) ABSTRACT

The invention provides methods of sample identification using germline structural variants to assess sample contamination and sample swap.

### FIELD OF THE INVENTION

The invention relates to methods of sample identification using structural variants.

### BACKGROUND

Sample swap and contamination present a challenge in molecular diagnostics. For example, in the detection of minimal residual disease (MRD), sample swap may result in false-negative results for some patients and false-positive results for others. In typical laboratory settings, hundreds or even thousands of samples may be present and tested daily. There is a high probability that sample swap and/or contamination may yield inaccurate results.

Conventional methods for detecting sample swap and contamination typically rely on generating single nucleotide polymorphism (SNP) profiles from a sample and comparing the results to subsequently-analyzed samples to confirm sample identity. Sample swap and contamination are detected by looking for unique combinations of SNPs in a sample. Typically, SNP profiles are based on the zygosity of the sample (which can be homozygous or heterozygous for a given SNP). Given that a maximum of 4 bases (A, T, C, G) may be represented in any given SNP (either in homozygosity or heterozygosity), the discriminatory power of SNPs relies on the combination of several SNP candidates. Conventional SNP arrays allow for the detection of thousands of SNPs. Typical SNP profiling performs well when hundreds or thousands of targets are analyzed by, for example, whole genome sequencing (WGS), but are limited when other techniques are used. For example, in PCR where the number of SNPs is divided by well or detection channel, there is limited discriminatory power even if the amount of input material is increased.

Contamination between samples adds a layer of complexity to the analysis of SNP arrays. DNA contamination may be suspected and can be detected from sequencing reads by modeling the likelihood of sequence reads as a mixture of two samples and estimating the fraction of reads from the contaminating sample. In addition, SNPs are often shared among many individuals. This means a large number of SNPs would need to be tracked in order to find a sequence unique to any particular individual. Finally, contamination is extremely hard to detect because it requires quantification of each SNP variant in a sample.

Finally, in the field of MRD every target molecule is of extreme importance. The allocation of some of this material to sample identification significantly reduces sensitivity. There exists, therefore, a need for a method that combines both sample analysis and sample profiling to prevent sample swap and contamination.

### SUMMARY

The present invention provides improved methods of sample identification and validation. Methods of the invention utilize structural variants to track tissue or body fluid samples and to identify sample swap. According to the invention, structural variants are identified in a sample and compared to a database containing known structural variants.

Structural variants (SVs) are genomic alterations/rearrangements in DNA. These alterations can involve copy number variation (deletions or duplications) or other events such as insertions, inversions, or translocations. SVs occur in both germ and somatic cells and are useful to understand healthy variations versus those associated with disease, such as cardiovascular diseases, neurological disorders, and cancer. A particular type of SV useful in methods of the invention are germline structural variations (GSV).

According to the invention, the frequency of an SV in a general population is useful as a genetic marker, allowing one to draw relationships between different populations. Accordingly, the invention allows SVs to confirm that a patient sample matches a previously analyzed and profiled sample. In one embodiment, sequences from tumor DNA samples are screened for the presence of germline SVs and compared to a database that contains genomic rearrangements in a population.

In one embodiment, the present invention allows for the selection of SVs based on population allele frequency, relatively low frequency in an internal patient cohort, presence in a diversity of chromosomes, or some combination thereof. Thus, preferred criteria for selection of SVs for use in the invention include low population allele frequency (no greater than about 10% in the overall population); lower frequency in an internal patient cohort; identification of unique germline fingerprints by processing through a bioinformatics pipeline; relative location of the SVs—for example on different chromosomes or at least separated on a single chromosome by a distance sufficient to create a distinct profile.

Following application of the above selection criteria, SVs are validated in both tumor DNA and matched non-tumor DNA (this can be buffy coat in the case of solid tumors or normal tissue samples). Validated GSVs in both tumor and non-tumor tissues are selected to be part of the SV fingerprint. In preferred embodiments, the SV is a GSV. In certain embodiments, the structural variants are derived from tumor biopsy, buffy coat, plasma, cfDNA, or ctDNA. The combination of structural variants may be detected by digital PCR, quantitative PCR, next-generation sequencing methods, or other methods known in the art.

According to methods of the invention, the same GSV fingerprint should be detectable in all samples derived from the same individual/patient, including tumor samples (if any) and normal (non-tumor) DNA samples, including cfDNA and ctDNA. If the GSV fingerprint is not detected in a sample, this confirms that a sample swap has occurred. By selecting rare (based on population frequency) GSVs, the invention provides a high degree of confidence that the same GSV fingerprint will not be detected on a different patient sample if a sample swap has occurred. Nevertheless, a particular GSV may be shared by one or more patients. However, methods of the invention significantly reduce the probability of two patients sharing the same GSV fingerprint (e.g., 2 up to 4 GSVs, 1 GSV, 2 GSVs, at least 100 GSVs). For instance, if 4 SNPs are used to distinguish samples, there is a 1 in 81 [1/(⅓\hat{ }4)] chance that a sample swap will not be identified (as the patients will have the same SNP profile). In the case of GSVs; if all 4 GSVs have an allele frequency of 10% (a typical criterion in methods of the invention), a sample match to a different sample will be 1 in 10,000 [1/( 1/10\hat{ }4)] samples. Thus, with GSVs, the chance of an exact match for a regular sample can be 1 In 8,000,000 (depending on the frequency of that GSV combination, as many of the GSVs selected are found in <10% allele frequency in the overall population).

According to aspects of the invention, the absence of one or more selected structural variants indicates that a sample swap or sample contamination has occurred.

In certain embodiments, the present invention comprises whole genome sequencing (WGS), whole exome sequencing, or any equivalent method to identify variants. In addition, samples may be any tissue or body fluid sample.

Additional aspects and advantages of the invention are apparent upon consideration of the following detailed description thereof.

### DETAILED DESCRIPTION

The invention provides improved methods for the detection and prevention of sample swap and contamination. Methods of the invention comprise identification of structural variants, especially germline structural variants (GSV), to detect sample swap and contamination. Sample SVs are compared to a panel of SVs from healthy tissue in consideration of population levels in order to identify sample contamination and/or the mixing of patient samples (sample swap).

Structural variants (SVs) are genomic alterations/rearrangements involving DNA segments. SVs occur in both germ and somatic cells and can be used to understand healthy variations as well as disease. Some SVs have been associated with cardiovascular diseases, neurological disorders, and even cancer.

The frequency of a same SV variation within a population can be used as a genetic mark allowing to draw relationships between different populations, a similar function as SNPs in population genetics.

### Structural Variant Selection and Sequencing

An exemplary method of the invention comprises sequencing DNA derived from a tumor sample and comparing sequences obtained against a database of known germline structural variants. GSVs that match between the sample and database entries are selected. A GSV can be any genomic variation including an insertion, a deletion, an inversion, a translocation, or the like. GSVs or combinations of GSVs may be detected by digital PCR, quantitative PCR, or other next-generation sequencing methods. GSVs can vary in size anywhere from about 2 bp to about over 10 kb, or even over 1 Mbs in length. In another embodiment, the method of the invention comprises sequencing DNA from a germline sample to detect GSVs.

GSVs from the tumor sequence are chosen to be a part of the unique patient sequence using a pre-selected criterion. For example, a low population allele frequency is desirable. Ideally, a selected GSV is observed in no more than 10% of the overall population. In one embodiment novel GSVs are selected from the sample (e.g., GSVs that have not been reported in the literature). GSVs present on different chromosomes are preferred. Alternatively, GSVs located further apart are preferred. As little as two GSVs may be chosen to become a part of the unique GSV fingerprint of a patient. However, the more GSVs chosen to become a part of the unique patient sequence, the more accurate the unique combination of GSVs are.

Selected tumor GSVs are then validated. GSVs present in both normal DNA and tumor DNA from the patient are included as identifying the patient. This means that the unique GSV fingerprint of a patient is present in any sample derived from that patient. If a sample is taken from that patient and the unique GSV fingerprint of a patient is not detected, this indicates a sample swap has occurred.

Sample contamination may also be detected using the unique GSV fingerprint of a patient. For example, if an expected GSV should be detected 1:1 within a reference region but, less than that appears in the sample, this suggests the sample has likely been contaminated.

The unique GSV fingerprint of a patient found using this process serves as a helpful and potentially life-saving reference point for everyone involved throughout the treatment process.

Structural variants may be detected in any feasible manner. One preferred method for detection of structural variants comprises selectively amplifying a target of interest in DNA derived from a sample. The pre-amplification step generates amplicons that include a copy of a variant of interest. The pre-amplification can, for example, use PCR reagents with primers designed to flank a variant of interest, increasing the abundance of copies (e.g., amplicons) that include the variant.

Once pre-amplification has been performed, the sample is partitioned and subject to a dPCR protocol that involves two stages, or types, of amplification. In the first stage of the dPCR, the amplicons are copied using variant-specific primers and tailed primers that operate to form tailed amplicons, which are then probed by dPCR. Methods of the invention have high sensitivity and are useful for discovering structural variants (SV) in samples including, for example, a tumor-specific structural variants in cfDNA in a blood or plasma sample. Due to that sensitivity, methods of the invention provide a useful and easy method for sample tracking after treatment. The variant-specific primer may preferably be a breakpoint-spanning primer that anneals substantially on one side of a breakpoint of a structural variant, but has at least a few bases at the 3′ end that anneal on the other side of the breakpoint. By using a breakpoint spanning primer, dPCR is expected, in the majority of situations, to only give a positive result for aqueous partitions that include a copy of the SV. Such methods comprise a an exponential amplification of the SV with the participation of a tailed primer and the universal primer. Annealing and hydrolysis of probes, which translates in the emission of fluorescent signal, also happens during the exponential phase.

Exemplary methods for SV detection include amplifying tailed amplicons with universal primers and detection probes that anneal to the tailed amplicons. Extension of the universal primers along the tailed amplicons through annealed detection probes generates a signal that shows the presence of the variant in the sample. For example, the probes may be fluorescently quenched hydrolysis probes that are digested by a 5′-3′ exonuclease activity of the polymerase. Thus, aqueous partitions that include amplicons with copies of the variant of interest will produce unquenched fluorophores and will fluoresce. The dPCR reaction volumes can be monitored by a fluorescence detector, a microscope, or other dPCR instrument to detect presence of the variant in the sample.

Pre-amplification of nucleic acid in a sample is useful to generate amplicons that include a copy of a selected variant. Then variant-specific primers are annealed and extending to create copies of the amplicon, which are then copied with tailed primers to form tailed amplicons. The tailed amplicons are amplified with universal primers and detection probes that anneal to the tailed amplicons. Extension of the universal primers along the tailed amplicons through annealed detection probes generates a signal that shows the presence of the variant in the sample. With respect to structural variants, the pre-amplification is performed with a PCR primer pair designed to anneal to sites that flank a breakpoint of the structural variant. Some methods may include partitioning the samples into aqueous compartments (e.g., droplets, microchambers, or wells of a plate) after the pre-amplification and performing the amplifying step as digital PCR in the aqueous compartments.

After a pre-amplification, the sample may be partitioned, and a first amplification performed with variant specific primers and tailed primers. The variant-specific primers may be breakpoint-spanning primers. The tailed primers generate tailed amplicons. The annealing and extending of the tailed primers and variant-specific primers may include thermocycling the amplicons in the presence of the variant-specific primers at temperatures that yields substantially only linear production of the copies. The variant-specific primers may be designed to anneal near, and be extended through, a variant of interest. Preferably, the variant-specific primers are designed to anneal to, and only be extended in the presence of, a variant of interest. The variant may be a polymorphism, a small indel, or a breakpoint of a structural variant.

The downstream amplifying steps may be performed together by thermocycling at temperatures that promote exponential amplification. For detection, a second amplification is performed on the tailed amplicons in the presence of a detection probe. The detection probe may be a hydrolysis probe that anneals to the tail and is digested by exonuclease activity of a polymerase used for the amplifying step. The detection probes may be universal probes that anneal to a universal probe binding site on a tail of the tailed amplicons.

The polymerase used in the preamplification may need to be inactivated after the pre-amplification. The inactivating step may be performed with a thermolabile proteinase, and the method may include heat denaturing the proteinase prior to the dPCR amplifying steps. The amplification may be performed with no sample cleanup between steps.

Amplification methods as set forth herein may be performed with multiplexed primer and probe sets that amplify and detect multiple distinct structural variants of interest. The detection probes may include one or more universal primer that anneals to a universal binding site on a tail of the tailed amplicons. In some embodiments, identical copies of the universal probe can be used simultaneously in a single dPCR assay to detect the multiple distinct variants of interest. In some embodiments, multiple distinct GSVs are detected in a single sample. In further embodiments, the sample is in a single well or several wells.

What is claimed is:

1. A method of sample identification comprising:
sequencing a tumor and detecting for the presence of one or more structural variants;
comparing the structural variants of the sequenced tumor to one or more databases which contain known structural variants;
selecting a combination of structural variants from the tumor to serve as a unique patient identifier; and
analyzing a patient sample for the selected combination of structural variants.

2. The method of claim 1, wherein the structural variant is a germline structural variant (GSV).

3. The method of claim 1, wherein the structural variant is a deletion, a duplication, an insertion, an inversion, or a translocation.

4. The method of claim 1, wherein the one or more structural variants are greater than 2 bp.

5. The method of claim 1, wherein the one or more structural variants are greater than 10 kb.

6. The method of claim 1 wherein structural variants are selected based on low population allele frequency, lower frequency in an internal patient cohort, presence in a diversity of chromosomes, or some combination thereof.

7. The method of claim 6, wherein the population allele frequency is less than about ten percent.

8. The method of claim 1, wherein the one or more structural variants are derived from tumor biopsy, buffy coat, plasma, cfDNA, or ctDNA.

9. The method of claim 1, wherein the combination of structural variants are detected by digital PCR, quantitative PCR, or other next-generation sequencing methods.

10. The method of claim 1, wherein the absence of one or more selected structural variants indicates that a sample swap or sample contamination has occurred.

11. The method of claim 1, wherein the tumor is sequenced by whole genome sequencing (WGS), whole exome sequencing, or any equivalent method.

12. The method of claim 1, wherein one or more methods of tumor sequencing are conducted on the same sample.

13. The method of claim 1, wherein the patient sample is any bodily sample which contains normal DNA.

14. The method of claim 1, wherein the combination comprises from 1 to 5 structural variants.
